(19) United States
(12) Patent Application Publication
(10) Pub. No.: US 2005/0130129 A1
(43) Pub. Date: Jun. 16, 2005

(54) Targeting gene transfer vectors to certain cell types by pseudotyping with viral glycoprotein

(76) Inventors: Gary J. Nabel, Bethesda, MD (US); Rafael Delgado, Ann Arbor, MI (US); Zhi-yong Yang, Potomac, MD (US)

Correspondence Address: LAHIVE & COCKFIELD, LLP., 28 STATE STREET, BOSTON, MA, 02109, US

(21) Appl. No.: 10/860,878

(22) Filed: Jun. 3, 2004

Related U.S. Application Data
(63) Continuation of application No. 09/600,766, filed on May 14, 2001, now abandoned.
(60) Provisional application No. 60/072,033, filed on Jan. 21, 1998.

(51) Int. Cl. C12Q 1/70; C12Q 1/68; C12N 7/00; C12N 15/867; C07K 14/005

(52) U.S. Cl. 435/5; 435/6; 435/456; 435/235.1; 530/395; 435/458

(57) ABSTRACT

The present invention provides compositions and methods for targeting gene transfer vectors to certain cell types by pseudotyping with a transmembrane form of viral glycoprotein, such as that from Ebola virus. The methods comprise the step of administering to a cell population a gene to be transferred operatively linked to an appropriate transfer vehicle, wherein the transfer vehicle is associated with a transmembrane form of viral glycoprotein.

### FIELD OF THE INVENTION

The present invention relates generally to compositions and methods for selective gene transfer, and in particular, to methods for targeting genes to certain cell types, comprising introducing to a cell population the gene to be transferred operatively-linked to an appropriate transfer vehicle, wherein the transfer vehicle is associated with a transmembrane form of viral glycoprotein.

### BACKGROUND OF THE INVENTION

Ebola virus has been identified as the cause of several highly lethal outbreaks of hemorrhagic fever. Infection begins typically with flu-like symptoms which often progress rapidly to fatal complications of hemorrhage, fever, and hypotensive shock. Bowen, E. T. W. et al., Lancet 1:571 (1977); Centers for Disease Control, M.M.W.R. 44:381 (1995); Le Guenno, B. et al., Lancet 345:1271 (1995); Peters, C. J. et al., Fields Virology, B. N. Fields, D. M. Knipe and P. M. Howley, Eds. (Lippincott-Raven, Philadelphia) p.1161 (1996). The negative-stranded genome of Ebola virus contains seven structural and regulatory proteins (Sanchez, A. et al., Virus Res. 29:215 (1993)), but despite its relative simplicity, the molecular basis for Ebola virus pathogenicity is unknown. Among the viral gene products, the glycoprotein is found in two forms: a secreted form, 50-70 kD (Sanchez, A. et al., PNAS (USA) 93:3602 (1996)), synthesized at high levels early in the course of infection, and an alternative transmembrane form, which arises from RNA editing to encode a 120-150 kD glycoprotein that is incorporated into the virion. Sanchez, A. et al., PNAS (USA) 93:3602 (1996); Volchkov, V. E. et al., Virology 214:421 (1995). The first 295 amino acids (aa) of both proteins are identical in the Zaire strain, while sGP contains an additional 69 and GP another 381 COOH— terminal aa residues. Sanchez, A. et al., PNAS (USA) 93:3602 (1996). The specific cellular targets of these related gene products and their roles in the pathogenesis of Ebola infection have not been characterized.

### SUMMARY OF THE INVENTION

The present invention provides compositions and methods for targeting gene transfer vectors to certain cell types by pseudotyping with a transmembrane form of viral glycoprotein. In one embodiment, the methods of the invention comprise the step of administering to a cell population a gene to be transferred operatively-linked to an appropriate transfer vehicle, wherein the transfer vehicle is associated with a transmembrane form of Ebola glycoprotein. In this embodiment, the gene will be targeted to cell types naturally infected with Ebola such as endothelial cells, monocytes and hepatocytes.

Genetic constructs for selective gene transfer into certain cell types are also provided. The genetic constructs of the present invention comprise a gene to be transferred operatively-linked to an appropriate transfer vehicle or carrier, wherein the transfer vehicle or carrier is associated with a transmembrane form of viral glycoprotein. In one embodiment, the transmembrane form of Ebola glycoprotein is expressed on the surface of a virus-based gene-targeting vector, e.g., lentiviral or retroviral vector. In another embodiment, an expressed or synthesized transmembrane glycoprotein is chemically derivatized to a non-biologic gene targeting vehicle.

Additional objects, advantages, and features of the present invention will become apparent from the following description and appended claims, taken in conjunction with the accompanying drawings.

### BRIEF DESCRIPTION OF THE DRAWINGS

The various advantages of the present invention will become apparent to one skilled in the art by reading the following specification and subjoined claims and by referencing the following drawings.

FIGS. 1A-1C show the binding of sGP to neutrophils;

FIGS. 2A-2D show the infection of different cell types by a GP-pseudotyped vector of the present invention;

FIGS. 3A-3F show the dependence of sGP binding on CD 16b and correlation of binding with neutrophil activation;

FIGS. 4A-4B show the effect of sGP on neutrophil function;

FIGS. 5A-5C show the infection rate of cells with a GP-pseudotyped retroviral vector of the present invention;

FIG. 6 is a schematic of the plasmid pVR 1012-GP(IC) (Ivory Coast strain of GP, see SEQ ID NO: 1);

FIG. 7 is a schematic of the plasmid pVR 1012-GP(S) (Sudan strain of GP, see SEQ ID NO: 2);

FIG. 8 is a schematic of the plasmid pVR 1012-GP(Z) (Zaire strain of GP, see SEQ ID NO: 3);

FIG. 9 is a schematic of the plasmid pVR 1012-sGP(Z) (Zaire strain of sGP, see SEQ ID NO: 4); and

FIG. 10 is a summary of the characterization of GP and sGP derivatives for their ability to pseudotype to induce cytotoxicity in producer cells.

### DETAILED DESCRIPTION OF THE PREFERRED EMBODIMENTS

The present invention provides genetic constructs and methods for targeting gene transfer vectors to certain cell types by pseudotyping with a transmembrane form of viral glycoprotein. The methods for selective gene transfer of the present invention comprise the step of administering to a cell population a genetic construct of the present invention so that the gene is transferred and expressed in certain cell types present in the cell population. Administration to the cell population may be ex vivo or in vivo.

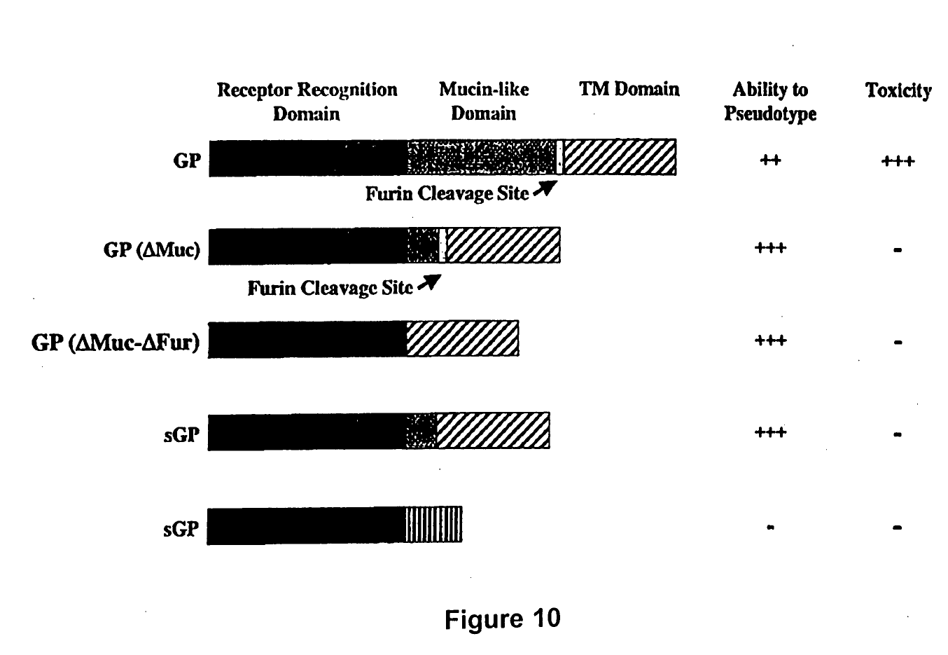

The genetic constructs of the present invention comprise a gene to be transferred operatively-linked to an appropriate transfer vehicle or carrier, wherein the transfer vehicle or carrier is associated with a transmembrane form of viral glycoprotein. In one embodiment, the transmembrane form of Ebola glycoprotein is associated with the vehicle or carrier. The gene to be transferred will thus be targeted to cell types naturally infected with Ebola virus including endothelial cells, hepatocytes, monocytes and related cell types such as dendritic cells. The transmembrane form of Ebola glycoprotein may be chosen from, without limitation, the Ivory Coast strain (SEQ ID NO: 1), Sudan strain (SEQ ID NO: 2), the Zaire strain (SEQ ID NO: 3) and/or the Reston strain. It will be appreciated that in other embodiments of the present invention, other hemorrhagic fever virus glycoproteins, in particular transmembrane glycoproteins, may be employed and will target those cell types naturally infected by the virus. Examples of hemorrhagic viruses include dengue virus, Yellow Fever virus (flaviviridae); Lassa, Junin and Machupo (arenaviridae); Rift Valley, Congo-Crimean and Hantaan (bunyaviridae); and Marburg (filoviridae). It will also be appreciated that derivatives of the transmembrane glycoprotein which retain the capability of targeting specific cell types, may also be employed, for example, the transmembrane glycoproteins may be mutated, e.g., toxic regions may be removed to improve producer cell viability (see FIG. 10).

The transmembrane glycoprotein may be expressed on the surface of a virus-based gene-targeting vector, e.g., lentiviral, retroviral, replication-deficient retroviral, adenoviral or adeno-associated viral vector. The transmembrane glycoprotein may also be expressed or synthesized and chemically derivatized to a non-biologic gene targeting vehicle, e.g., liposome or DNA-protein complex.

The term “operatively-linked” as used herein refers to functional linkage between a nucleic acid expression control sequence (such as a promoter) and a second nucleic acid sequence (i.e., gene), wherein the expression control sequence directs transcription of the nucleic acid corresponding to the second sequence. Expression control sequences are known to those skilled in the art (see, e.g., Goeddel, Gene Expression Technology: Methods in Enzymology 185, Academic Press, San Diego, Calif. (1990)). “Associated with” as used herein refers to the transmembrane form of viral glycoprotein being in contact or linkage with the transfer vehicle or carrier in such a way as to direct the transfer vehicle or carrier to certain cell types. The terms “transfer vehicle” and “carrier” refer to any type of structure which is capable of delivering the gene of interest to a target cell.

Many transfer vehicles or carriers are known in the art. For example, various viruses that are capable of infecting cells can be recombinantly manipulated to carry the gene of interest without affecting their infectivity. As used herein, the terms “infect” and “infectivity” refer only to the ability of a virus to transfer genetic material to a target cell. Those terms do not mean that the virus is capable of replication in the target cell. In fact, it is preferable that such viruses are replication defective so that target cells do not suffer the effects of viral replication.

In one embodiment, the virus employed is a replication defective retroviruses. When these replication defective retroviruses are employed, their genomes can be packaged by a helper virus in accordance with well-known techniques. Suitable retroviruses include PLJ, pZip, pWe and pEM, each of which is well known in the art. Suitable helper viruses for packaging genomes include ψCrip, ψCre, ψ2, ψAm and adeno-associated viruses.

In another embodiment, lentiviral vectors are employed. Surprisingly, the inventors of the present invention were successful in pseudotyping lentiviral vectors (HIV) with the transmembrane glycoprotein from Ebola. Feline immunodeficiency virus, bovine immunodeficiency virus, simian immunodeficiency virus and EAIV, may also be employed as the carrier in the compositions and methods of the present invention.

Gene delivery systems other than viruses may also be employed. For example, the gene to be transferred may be packaged in a liposome which is chemically derivatized to the transmembrane glycoprotein. To form these liposomes, one mixes the DNA of an expression vector which expresses the gene to be transferred with lipid, such as N-[1-(2,3,-dioleyloxy)propyl]-N,N,N-trimethylammonium chloride (DOTMA) in a suitable buffer, such as Hepes buffered saline. This causes the spontaneous formation of lipid-DNA complexes (liposomes). Felgner, P. L. et al., PNAS (USA) 84:7413-7417 (1987).

Another gene delivery system that may be utilized in this invention is DNA-protein complexes. The formation of DNA-protein complexes is described in U.S. Pat. No. 5,166,320, the disclosure of which is herein incorporated by reference.

It will be appreciated that any gene may be employed in the compositions and methods of the present invention. For example, and without limitation, in the treatment of cancer, death inducing genes, including genes coding for cytostatic or cytotoxic proteins, e.g., HSV tk, and genes encoding cyclin dependent kinase inhibitors, p21, p27, cytosine deaminase, and fas ligand, may all be employed. In another example, for the treatment of cardiovascular or ischemic vascular disease, genes encoding angiogenic factors such as VEGF basic or acidic FGF's (FGF 1-5) may be employed. In yet another example, in the treatment of vasospasm, the gene encoding NO synthase or heme oxygenase, may be employed. In a further example, monocytes and dendritic cells may be targeted with genes encoding immunogens for cell-targeted immunization.

In one embodiment, the methods of targeting gene transfer vectors to certain cell types involve administering to a cell population ex vivo, a construct of the present invention and introducing the transfected cells into a subject. In an alternative embodiment, the methods of the present invention comprise administering to an in vivo cell population a construct of the present invention. Administration can be by any of the routes normally used for in vivo gene therapy such as direct delivery to cells via a gene gun, and other known techniques. The constructs are thus administered in any suitable manner, preferably with pharmaceutically acceptable carriers. The constructs can be administered, for example, by intravenous infusion, orally, topically, intraperitoneally, intravesically or intrathecally. The preferred method of administration will often be intravenous.

To practice an ex vivo method of the present invention, a source of cells is obtained. The cells are optionally selected from in vitro cells, such as those derived from cell culture and ex vivo cells, such as those derived from a subject. The term “subject” is intended to include living organisms, e.g., mammals. Examples of subjects include humans, primates, dogs, cats, mice, rats, and transgenic species thereof. It will be appreciated that specific cell populations may be obtained by isolation from certain tissues by methods known to those skilled in the art. The cells are maintained under conditions necessary to support growth, for example an appropriate temperature (e.g., 37° C.) and atmosphere (e.g., air plus 5% CO₂).

The cells are then transfected with the constructs of the present invention by introducing the constructs to the cell population, under conditions favorable for transfection. According to one embodiment of the present invention, cells are treated with compounds that facilitate uptake of the constructs by the cells. According to another embodiment of the present invention, cells are treated with compounds that stimulate cell division and facilitate uptake of the constructs. It will be appreciated that compounds that facilitate uptake of constructs by cells and compounds that stimulate cell division are known to those skilled in the art.

The constructs of the present invention express the transferred gene in a dose dependent manner. The specific dose to be administered to a patient will be determined by the efficacy of the particular construct and/or delivery system employed, the gene transferred, and the condition of the patient, as well as the body weight or surface area of the patient to be treated. The size of the dose also will be determined by the existence, nature, and extent of any adverse side-effects that accompany the administration of a particular construct or effect a particular patient. In determining the effective amount of the construct or transfected cell to be administered, the physician needs to evaluate circulating plasma levels, toxicities, and progression of disease. It will be appreciated that administration can be accomplished via single or divided doses.

There is a wide variety of suitable formulations for pharmaceutical compositions containing the constructs of the present invention. Formulations suitable for oral administration can consist of (a) liquid solutions, such as an effective amount of the construct dissolved in diluents, such as water, saline or PEG 400; (b) capsules, sachets or tablets, each containing a predetermined amount of the construct, as liquids, solids, granules or gelatin; (c) suspensions in an appropriate liquid; and (d) suitable emulsions. The construct, alone or in combination with other suitable components, may also be made into aerosol formulations to be administered via inhalation, e.g., to the bronchial passageways. Aerosol formulations can be placed into pressurized acceptable propellants, such as dichlorodifluoromethane, propane, nitrogen, and the like.

Suitable formulations for rectal administration include, for example, suppositories, which consist of the construct with a suppository base. Suitable suppository bases include natural or synthetic triglycerides or paraffin hydrocarbons. In addition, it is also possible to use gelatin rectal capsules which consist of a combination of the construct with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules or vials. Extemporaneous injection solutions and suspensions can be prepared from sterile powders, granules, and tablets of the kind previously described. Cells transfected by the constructs as described above in the context of ex vivo therapy can also be administered as described above.

This invention also provides compositions and kits comprising the constructs of the present invention. For example, the composition can comprise the constructs of the present invention in a pharmaceutically acceptable carrier as described above. Kits comprising such compositions and instructions for use are also within the scope of this invention.

In order to more fully demonstrate the advantages arising from the present invention, the following examples are set forth. It is to be understood that the following is by way of example only and is not intended as a limitation on the scope of the invention.

### SPECIFIC EXAMPLE 1

I. Methods

Recombinant retroviruses were produced by transient transfection of 293T cells: 2×10⁶ cells were plated 24 hours before transfection in 60 mm dishes. Transfection was performed by calcium-phosphate precipitation using 3 μg of a retroviral vector (Kinsella, T. M. et al., Hum. Gene Ther. 7:1405 (1996)) encoding lucerifase linked to an internal ribosome entry site and a green fluorescent protein derivative (GFP; pEGFP, Clontech), pLZRs-Luc-Gfp, 5 μg of an expression vector encoding gag and pol, pNGVL-MLVgag-pol, and 1 μg of the envelope encoding plasmid: pNGVL4070A (ampho) env, pCMV-Eco env or p1012-Ebola GP, respectively. Supernatants corresponding to 24-48 hours post-transfection were harvested, cleared by low-speed centrifugation and either used immediately for infection or frozen at −80° C. Infections were performed in 6-well plates (1.5-2.5×10⁵ adherent cells) or 12-well plates (5×10⁵ non-adherent) using different dilutions of the supernatants by incubating the cells overnight with 1 ml and 300 μl, respectively of the diluted supernatants. Polybrene was used at a concentration of 5 μg/ml for all the cell lines except for D17 in which the concentration was 100 μg/ml. After overnight infection, fresh medium was added and the cells were incubated for an additional 24 hours. After infection, the cells were lysed in 25 mM Tris-phosphate pH 8, 2 mM DTT, 2 mM 1,2-diaminocyclohene-N,N,N′,N′-tetraacetic acid, 10% glycerol, 1% TritonX-100, and assayed for luciferase activity using Luciferase Assay Reagent (Promega, Madison, Wis.) in a 1251 BioOrbit Luminometer. The same number of cells (range 5-10×10⁴) was analyzed for every specific cell line.

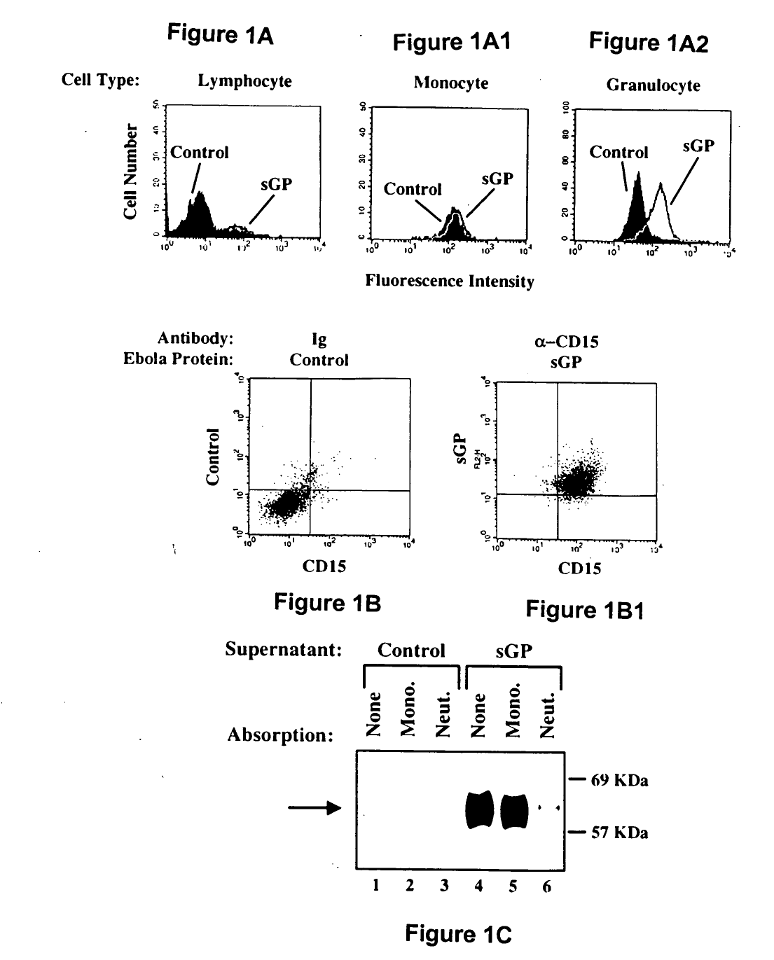

Binding of sGP to neutrophils and inverse correlation of binding with activation: FIGS. 1A-1A2. PBMC from normal volunteers were incubated with control or sGP supernatants derived from transfected 293 cells, and immunostaining was performed using a rabbit antibody to sGP as previously described. Sanchez, A. et al., PNAS (USA) 93:3602 (1996); Xu, L. et al., Nat. Med. (1997) in press. Secondary staining was performed with a fluorescein isothiocyanate (FITC)-conjugated goat anti-rabbit IgG antibody (Sigma, F9887). All incubations were performed at 4° C. for 30 minutes with 0.4 μg of the relevant antibodies per 10⁶ cells in a 50 μl volume.

FIGS. 1B-1B1. Double immunostaining with antibodies to sGP and the neutrophil-specific marker, CD15. Cells were incubated with a FITC conjugated mouse anti-human CD15 antibody (Caltag, cat# MHCD1501), followed by secondary staining with a PE-conjugated anti-rabbit IgG antibody (Sigma) to detect sGP binding. Cells were washed with PBS, fixed in 1% formaldehyde, and analyzed by FACS.

FIG. 1C. Specific absorption of sGP by neutrophils. Control or sGP supernatants derived from relevant transfected 293 cells (Xu, L. et al., Nat. Med. (1997) in press) were incubated at 1:500 dilution with 10⁶ mononuclear or granulocytic cells. Cells were removed and the resulting supernatants analyzed by an 8% SDS PAGE gel. Western blot analysis was performed as previously described (Xu, L. et al., Nat Med. (1997) in press) using an anti-GP rabbit antisera and a secondary antibody, horseradish peroxidase conjugated donkey anti-rabbit IgG at a dilution of 1:5,000 (Amersham, NA934). Primary antibody was incubated for 30 minutes at room temperature, as was the secondary antibody. The immunocomplexes were detected by chemiluminescence using Supersignal® chemiluminescent substrate reagents (Pierce) according to the manufacturer's instructions. Arrow indicates sGP reactive band.

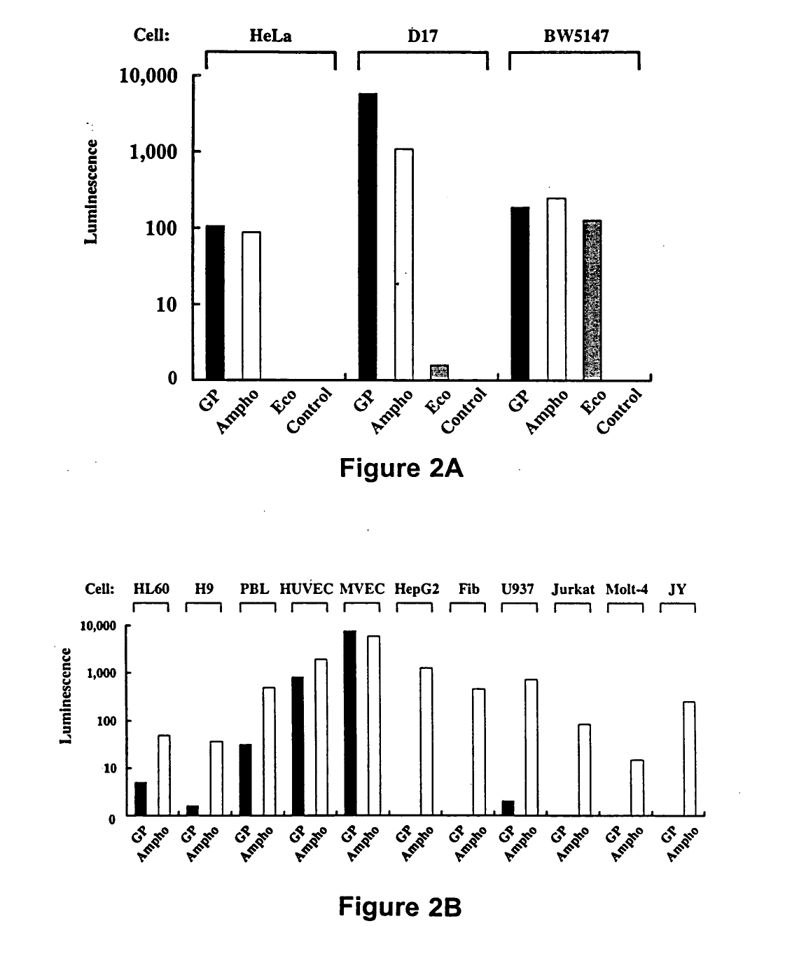

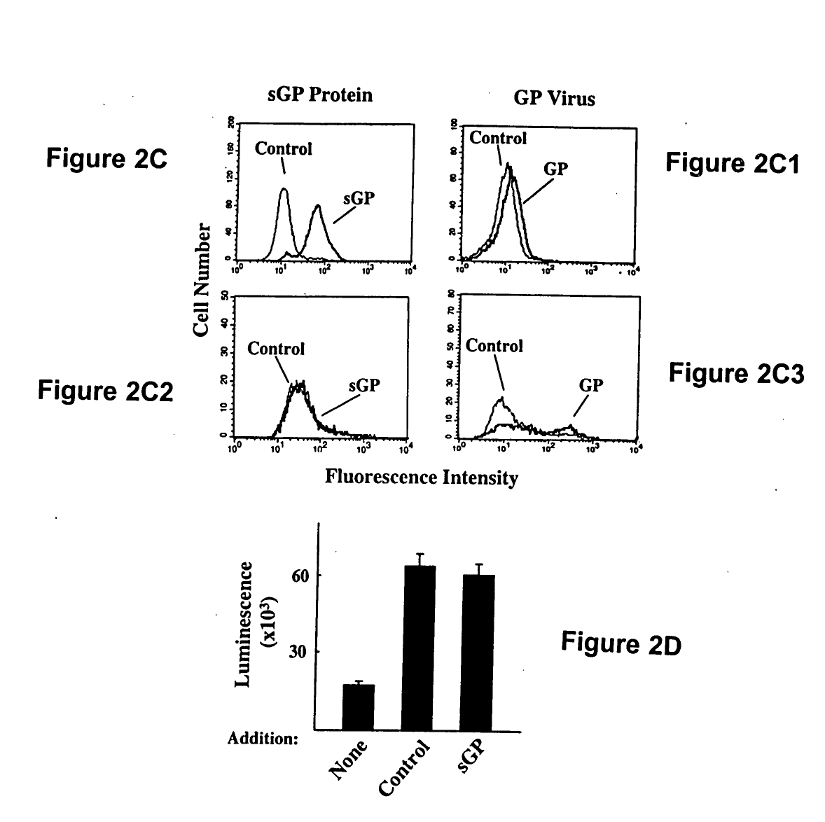

Infection of different cell types by GP-pseudotyped retroviral vector and preferential binding to endothelial cells: FIG. 2A. Infection of different indicator cell lines with the Ebola-GP pseudotyped retrovirus expressing luciferase. Amphotropic and ecotropic retroviral vectors were used as controls. Viruses were diluted to different multiplicities of infection (MOI) to provide for equal luciferase activity on Hela cervical epithelial cells, permissive for amphotropic retrovirus, D17 dog osteosarcoma cells (Embretson, J. E. et al., J. Virol. 61:3454 (1987)), which are permissive for amphotropic, xenotropic, and ecotropic retroviruses, and BW5147 T leukemia cells permissive for amphotropic and ecotropic virus. In these groups, GP virus titer was 1-4×10⁵/ml and amphotropic virus was ˜2×10⁴/ml (MOI≈1.0 and 0.1, respectively), and the ecotropic virus titer was ˜10⁶/ml (MOI≈10). Titers were determined by endpoint dilution of reporter activity of the amphotropic virus in D17 cells, and was normalized to reverse transcriptase activity for the GP virus.

FIG. 2B. Analysis of different normal or transformed cell lines by infection with amphotropic or GP retroviral vectors at the same titer (10⁴/ml, MOI≈0.2). Forty-eight hours after infection, an equivalent of 5×10⁴ cells was assayed for luciferase activity after exposure to equal titers of viral stocks. Luminescence is expressed as the fold-increase over non-infected control cells.

FIGS. 2C-2C3. The binding of sGP (left) or GP-pseudotyped retrovirus (right) to neutrophils (upper panel) or microvascular endothelium (lower panel) was determined by FACS. sGP binding was performed as in FIG. 1A, and retrovirus incubation was performed at 37° C. for 2 hours in the presence of polybrene (8 μg/ml).

FIG. 2D. Infection of D17 cells by GP-pseudotyped virus in the absence (lane 1, none) or presence of control (lane 2) or sGP supernatant (lane 3) from transfected 293 cells. Gene transfer was measured by the luciferase assay as described below. Luminescence refers to relative light units in the luciferase assay.

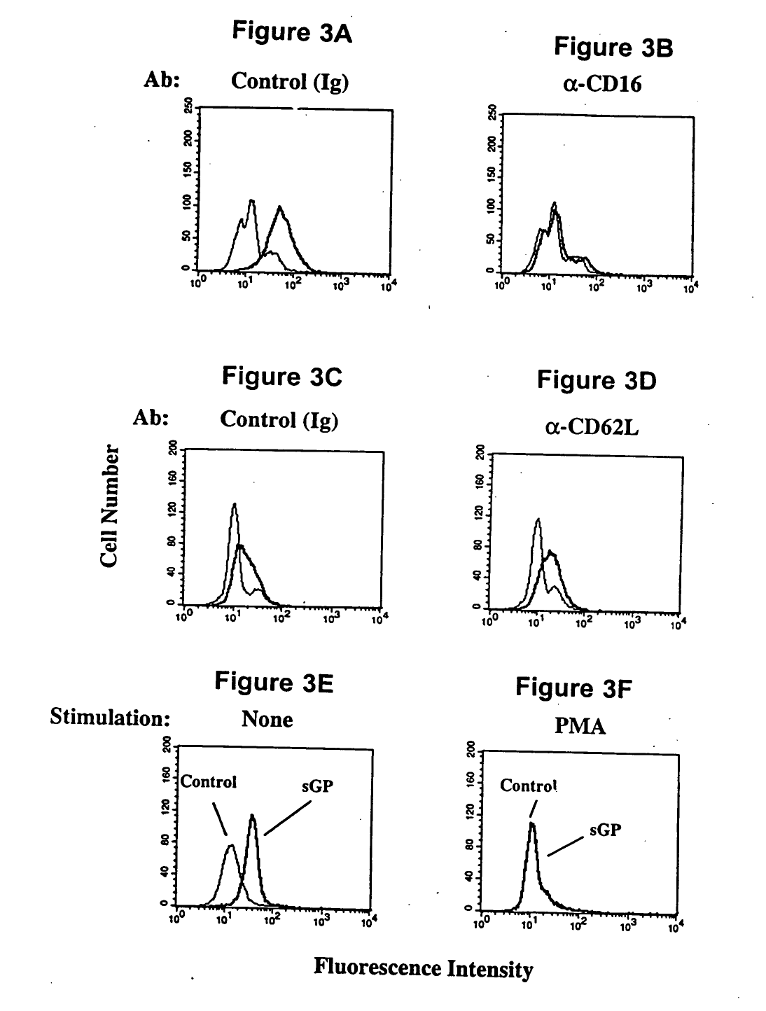

Dependence of sGP binding on CD16b and correlation of binding with neutrophil activation: FIGS. 3A-3D. Neutrophils were incubated for 30 minutes at 4° C. with a mouse antibody to CD16b (upper panel; clone 3G8 from Immunotech, cat# 1M0813) or CD62L (middle panel, R&D Systems), compared to the indicated control antibody [purified mouse IgG (Vector Laboratories), 1-2000], followed by supernatants from control or sGP-transfected 293 cells, primary rabbit antibody to sGP, and a FITC-conjugated secondary antibody to rabbit IgG (FIG. 1, legend). Cells were washed with PBS, fixed in 1% formaldehyde, and analyzed by FACS. For blocking, 10⁶ cells were incubated with 0.5-1 μg of the relevant antibodies for 30 minutes in a 50 μl volume.

FIGS. 3E-3F. Immunostaining with sGP was performed on isolated neutrophils which were maintained in media (none) or incubated with PMA (10 ng/ml) at 37° C. for 30 minutes (PMA).

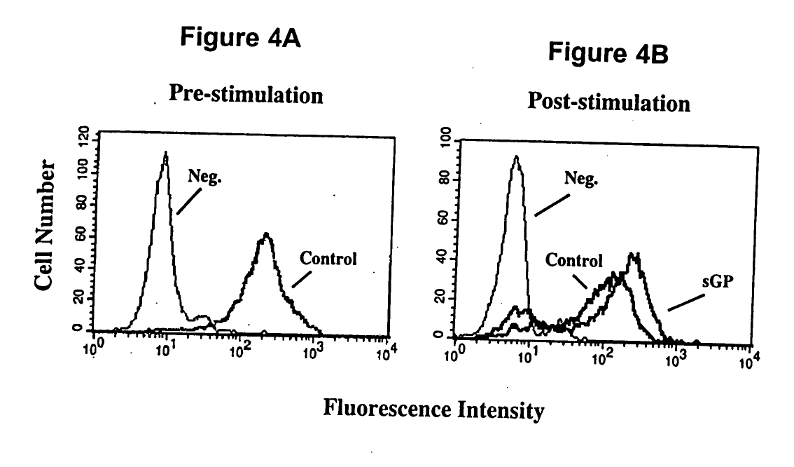

Effect of sGP on neutrophil function: FIGS. 4A-4B. Exposure of neutrophils to sGP inhibits down modulation of L-selectin. Isolated neutrophils were incubated with the indicated control or sGP containing supernatants (Xu, L. et al., Nat. Med. (1997) in press) and defined media (AIM V, GIBCO) for 4 hours at 37° C. Expression of L-selectin was determined using an anti-CD62L antibody (R&D Systems), followed by the secondary staining using a FITC-conjugated anti-mouse IgG (Sigma, F2883) as described in FIG. 1, legend. Cells were washed with PBS, fixed with 1 % formaldehyde and analyzed by FACS for relative levels of fluorescence intensity as a function of cell number. An isotype control was used to quantitate background levels of immunostaining (neg.). Results are representative of three independent experiments.

II. Results

To determine the specificity of Ebola virus glycoproteins, expression vectors encoding either sGP, GP, or a plasmid control (Xu, L. et al., Nat. Med. (1997) in press) were transfected into 293 cells, and cell culture supernatants were used as a source of relevant recombinant glycoproteins. Binding of sGP was determined by immunofluorescence analysis after incubation of relevant supernatants with normal or transformed human cell lines. No binding was detected to several hematopoietic lineages, including lymphocytes or monocytes (FIG. 1A), or transformed Jurkat or CEM T leukemias, the HL60 myelomonocytic or U937 promonocytic leukemia cells. In contrast, sGP was able to bind to granulocytic cells, as evidenced by FACS analysis of this subset of peripheral blood mononuclear cells (PBMC) discriminated by cell size and granularity (FIG. 1A). This cell specificity was confirmed by using double-staining with a granulocyte-specific cell surface marker, CD15 (FIG. 1B). Absorption of sGP by purified neutrophils in the absence of antibodies also resulted in depletion of sGP, indicating that binding to the neutrophil occurred in the absence of antibody (FIG. 1C).

A potential structural similarity between Ebola GP and avian sarcoma virus envelope protein has been previously proposed (Gallaher, W. R., Cell 85:477 (1996)), raising the possibility that this protein could be incorporated into retroviral particles. To determine the binding specificity of the transmembrane glycoprotein, pseudotyping of a Moloney leukemia virus was therefore attempted. Infectivity of different cell types by this pseudotyped vector was determined with a luciferase reporter gene. de Wet, J. R. et al., Mol. Cell. Biol. 7:725 (1987). This analysis revealed infection of cells different from those which interacted with sGP (FIG. 2A,B). For example, though it could infect other cell types, transduction by the GP retroviral vector readily occurred in endothelial cells, either from the microvasculature (MVEC) or umbilical veins (HUVEC) (FIG. 2B), which did not bind sGP (FIG. 2C, left). When the specificity of GP-retrovirus was compared to murine retroviruses pseudotyped with amphotropic or ecotropic envelope gp70 proteins, the range of susceptible target cells differed (FIG. 2B), suggesting that the virus receptor(s) for Ebola GP differ from those previously described for gp70. Minimal binding of GP-virus was observed on neutrophils, despite the ability of these cells to bind sGP (FIG. 2C, upper panel) and the fact that immunoreactive protein was detected on the virus. Conversely, GP-virus binding to endothelial cells was readily detected, though these cells did not bind sGP (FIG. 2C, lower panel). When sGP was analyzed for its effect on GP retroviral gene transfer, infection was not inhibited by sGP (FIG. 2D), further confirming the divergent specificities of the two forms of the viral glycoprotein. Recent studies have revealed that the biochemical forms of these proteins differ, with sGP present in solution primarily as a homodimer and GP as a trimer, suggesting that differences in multimer composition may contribute to these alternative specificities.

Potential cell surface receptors for sGP were analyzed with antibodies to several neutrophil cell surface antigens to interfere with sGP binding, including CD15, L-selectin (CD62L), CD16b, and several common leukocyte antigens. Only the neutrophil-specific form of the low affinity F_{c} yreceptor III, CD16b, inhibited sGP binding specifically. Antibodies to CD62L, for example, did not inhibit sGP binding (FIG. 3). Binding to neutrophils correlated with their activation state and CD16b expression since no binding was observed in cells stimulated with phorbol 12-myristate 13-acetate (PMA) for 30 minutes, at which time CD16b expression was markedly decreased on these cells (FIG. 3, lower panel). Overexpression of this form of CD16 on a heterologous cell type, 3T3 fibroblasts, did not confer sGP binding to these cells by FACS analysis, suggesting that CD16b is necessary but not sufficient for stable binding.

Binding of sGP did not inhibit neutrophil activation in response to potent pleiotropic activators (PMA, IL-8, or f-Met-Leu-Phe), as measured by down modulation of L-selectin expression using FACS analysis. In a defined serum-free medium, partial activation of neutrophils was observed, with a decrease in L-selectin expression at 4 hours (FIG. 4). Under these conditions, incubation of neutrophils with sGP supernatant prevented this decrease in L-selectin expression (FIG. 4). Because L-selectin was not required for sGP binding (FIG. 3), this effect was apparently indirect, through a mechanism not yet defined, possibly involving CD16b or carbohydrate interactions of the highly glycosylated sGP protein.

The expression of alternative Ebola virus glycoproteins in clinical infection has long been recognized, but their functional roles and cell specificity have not been defined. Early after infection, high levels of the secreted protein are found in the serum and precede fulminant replication and dissemination of virus systemically, at which time synthesis of trans membrane GP is markedly increased. Sanchez, A. et al., PNAS (USA) 93:3602 (1996). The inventors have now found that the binding specificities of these two molecules differ. It had been proposed that sGP may serve as a decoy to prevent recognition of GP, possibly to temporarily inhibit virus binding to target cells. The studies set forth herein suggest that this hypothesis is unlikely to be correct. The binding specificities of these proteins differ, and despite the fact that they are derived from the same viral gene, it has been surprisingly found that alternative forms of the glycoprotein have been selected for different functions.

Although these proteins share identical amino terminal sequences, their carboxyl terminal regions differ. Sanchez, A. et al., Virus Res. 29:215 (1993). These sequences are likely responsible for the differences in binding specificity, either through direct interactions in these domains or by their effect on multimerization. The secreted glycoprotein binds to neutrophils to prevent early events in activation, possibly serving to diminish any inflammatory responses which might provide innate immunity to the virus, facilitating productive viral replication. The subsequent increase in GP synthesis gives rise to virus which in turn could infect other cells. Filoviruses have been shown previously to infect and replicate in different cell types and appear to grow readily in endothelial cells in vivo. Peters, C. J. et al., Fields Virology, B. N. Fields, D. M. Knipe and P. M. Howley, Eds. (Lippincott-Raven, Philadelphia) (1996); Schnittler, H. J. et al., J. Clin. Invest 91:1301 (1993). The findings set forth herein suggest that its tropism for this cell type is probably determined by the specificity of GP. In Ebola infection, preferential binding and infection of microvascular endothelial cells may lead ultimately to a loss of capillary integrity that results in the severe hemorrhage observed in the terminal stages of this disease. The differential binding of these two gene products from the same viral structural gene generated by RNA editing suggests that they have evolved functionally to differentially affect immunity and infectivity. The ability to facilitate viral replication and target the virus to endothelial cells by alternative products of the same viral gene represents an efficient genetic mechanism which can account for different pathologic features of this disease. Inhibition of sGP binding to neutrophils and GP to endothelium is likely to ameliorate the effects of acute Ebola virus infection.

### SPECIFIC EXAMPLE 2

I. Methods

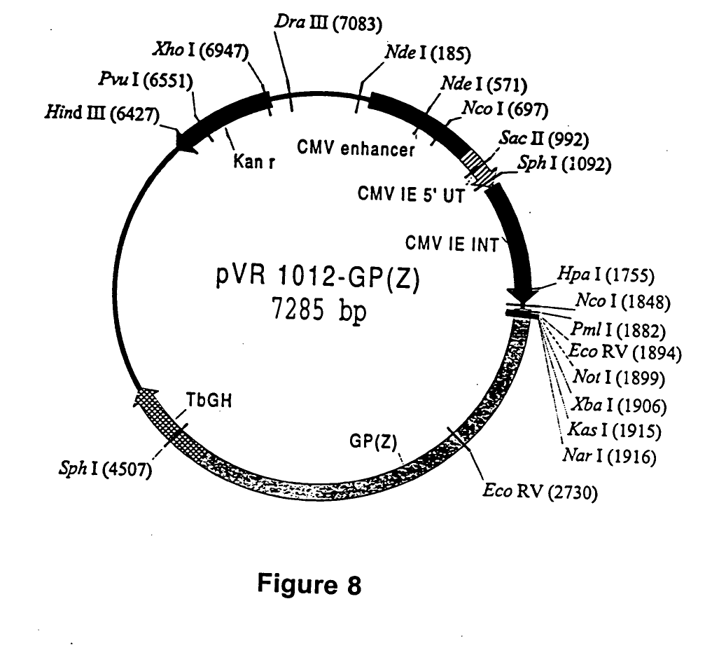

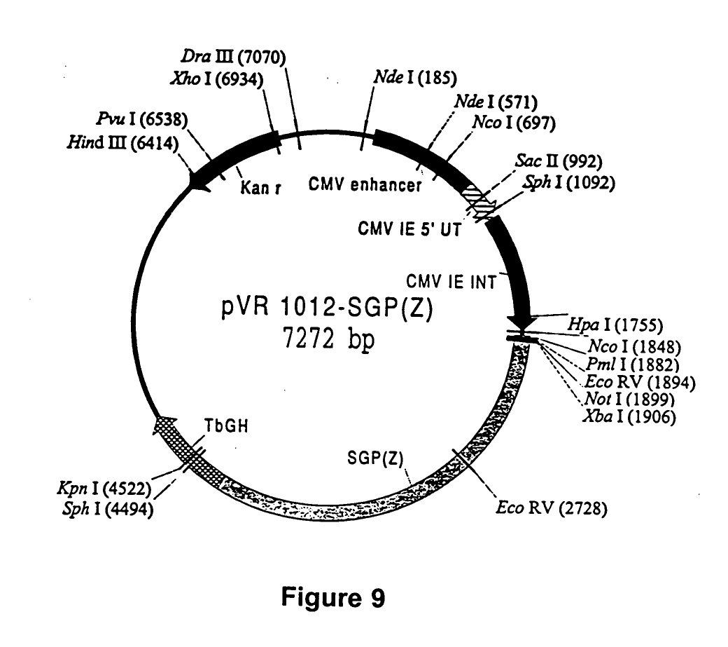

Production of pseudotyped MuLV retroviruses expressing green fluorescent protein (GFP): 50%-70% confluent 293 T cells in 60 mm tissue culture dishes were transfected using the calcium phosphate method and the following plasmids: 0.3 μg 1012 GP(Z) (see FIG. 8) or 1012 sGp-Gp(Z) (see FIG. 9), 3 μg LZR-gfp, 2 μg pNGVL-gag-pol. After overnight transfection, fresh media was added to cells. Twenty hours later, the supernatants were harvested and filtered through a 0.45 μm filter.

Infection of HUVEC cells using the pseudotyped retroviruses: The day before infection, 30%-50% confluent HUVEC cells were prepared in 6-well plates. 1 ml of pseudotyped retroviral supernatant was added to one well of the 6-well plates with 15 μg/ml of polybrene. Sixteen hours later, the viruses were removed and normal media was added. After 24 hours, the cells were lifted and GFP expression measured using FACS analysis.

Construction of 1012sGP-GP(Z): 1012 sGP(Z) cells were digested with Pstl and treated with Klenow, then digested with Xbal. 1012 GP(Z) cells were digested by EcoRI and treated with Klenow, then digested with Kpnl. Pstl/Klenow/Xbal treated sGP fragment and EcoRl/Klenow/Kpnl treated GP fragment were then cloned into Xbal/Kpnl treated pVR-1012 plasmid.

GP and sGP derivatives: The receptor recognition domain, mucin-like domain and/or TM domain of GP and sGP were mutated. The mutated GP and sGP was then tested for its ability to pseudotype and for cytotoxicity in producer cells.

II. Results

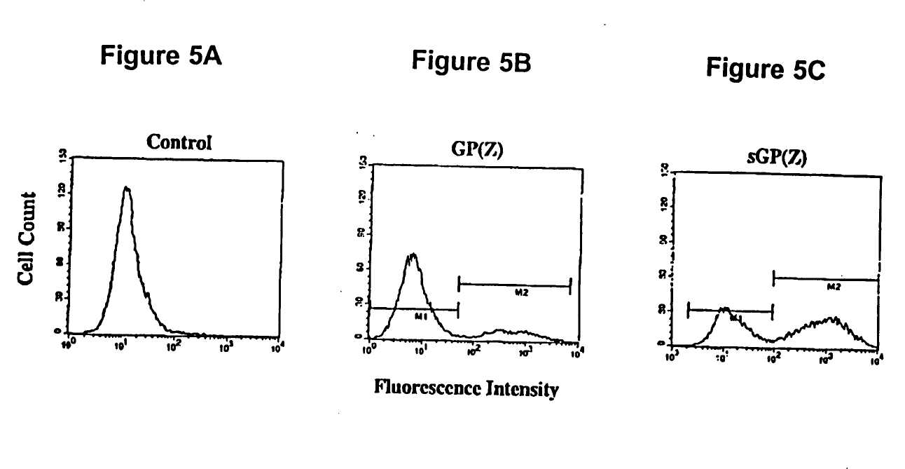

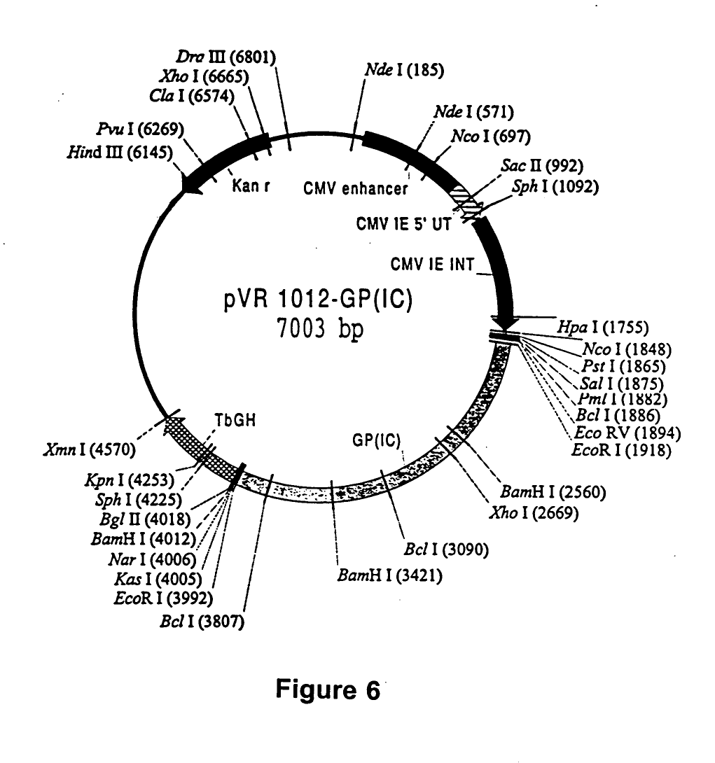

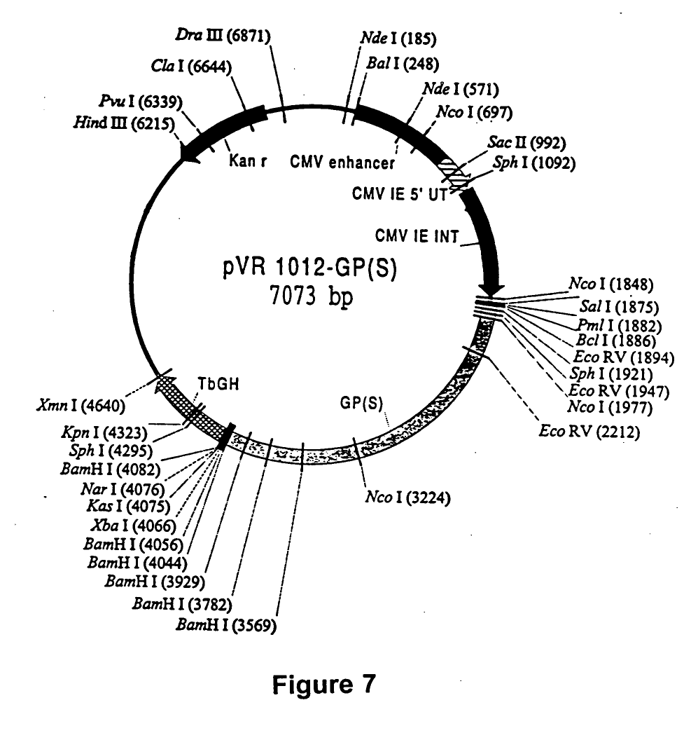

To determine the efficacy of targeting endothelium with the gene transfer vectors pseudotyped with GP of the present invention, HUVEC cells were infected with GP(Z) pseudotyped MuLV retrovirus (LZR-gfp) and sGP-GP(Z) pseudotyped MuLV retrovirus (LZR-gfp). FIGS. 5A-5C show the infection rate (GFP expression) measured using FACS analysis. As shown in FIG. 5B, the GP(Z) pseudotyped MuLV retrovirus (LZR-gfp) was effective in targeting and expressing GFP in endothelium.

To determine whether mutating GP would effect its ability to pseudotype and/or decrease toxicity in producer cells, the receptor recognition domain, mucin-like domain and/or TM domain were mutated. FIG. 10 shows the results. The optimal envelope is able to pseudotype but shows minimal toxicity.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

All patents and other references cited herein are incorporated by reference as if fully set forth.

1. A genetic construct comprising a gene operatively-linked to a carrier, wherein the carrier is associated with a transmembrane form of viral glycoprotein or derivative thereof.

2. The genetic construct of claim 1, wherein the transmembrane form of viral glycoprotein or derivative thereof is expressed on the surface of the carrier.

3. The genetic construct of claim 1, wherein the transmembrane form of viral glycoprotein or derivative thereof is from Ebola.

4. The genetic construct of claim 1, wherein the carrier is a viral vector.

5. The genetic construct of claim 1, wherein the carrier is a non-biologic gene targeting vehicle.

6. The genetic construct of claim 4, wherein the viral vector is a retroviral vector.

7. The genetic construct of claim 4, wherein the viral vector is a lentiviral vector.

8. The genetic construct of claim 5, wherein the non-biologic gene targeting vehicle is a liposome.

9. The genetic construct of claim 5, wherein the non-biologic gene targeting vehicle is a DNA-protein complex.

10. A method of targeting a gene to a cell comprising the step of administering to a cell population a genetic construct comprising the gene operatively-linked to a carrier, wherein the carrier is associated with a transmembrane form of viral glycoprotein or derivatives thereof.

11. The method of claim 10, wherein the transmembrane form of viral glycoprotein or derivative thereof is expressed on the surface of the carrier.

12. The method of claim 10, wherein the transmembrane form of viral glycoprotein or derivative thereof is from Ebola.

13. The method of claim 10, wherein the carrier is a viral vector.

14. The method of claim 10, wherein the step of administration is ex vivo.

15. The method of claim 10, wherein the step of administration is in vivo.

16. The method of claim 10, wherein the cell is an endothelial cell.

17. The method of claim 10, wherein the cell is a hepatocyte.

18. The method of claim 10, wherein the cell is a monocyte.

19. The method of claim 10, wherein the cell is a dendritic cell.

20. The method of claim 14, further comprising the step of introducing the cell population to a subject.


SEQUENCE LISTING (4 sequences)

SEQ ID NO: 1 - DNA, 7003 bp, Artificial Sequence
  Description of Artificial SequencePlasmid containing DNA forGP of Ebola Virus, Ivory Coast strain
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgcgcggccgctctagaattctctaatcacagtcatcatgggagcgtcagggattctgcaattgccccgtgagcgcttcaggaaaacatctttctttgtttgggtaataatcctattccataaagtcttttcaatcccgttgggggttgtacacaacaataccctacaagtgagtgatattgacaagtttgtgtgccgagacaaactctcttcaactagccaattgaagtcagtcgggttgaacttggagggcaatggagtagcaactgatgtaccaacggcaaccaaaagatggggttttcgagctggtgttccaccaaaggtggtaaattacgaagctggagaatgggctgagaactgttataacctggctataaagaaagttgatggtagtgagtgcctaccagaagcccctgagggagtgagggattttccccgttgccgctatgtacacaaagtctcaggaactggaccatgcccaggaggactcgcctttcacaaagaaggagccttcttcctgtatgaccgactcgcatcaacaatcatttatcggggtacaacctttgccgaaggagttattgcatttctgatcttgcctaaggcgcgaaaggattttttccagtctcctccattgcatgagcctgccaacatgaccacggatccctccagttactatcacacgacaacaataaactacgtggttgataattttggaaccaacaccacagagtttctgttccaagtcgatcatttgacgtatgtgcagctcgaggcaagattcacaccacaattccttgtcctcctaaatgaaaccatctactctgataaccgcagaagtaacacaacaggaaaactaatctggaaaataaatcccactgttgataccagcatgggtgagtgggctttctgggaaaataaaaaaacttcacaaaaaccctttcaagtgaagagttgtctttcgtacctgtaccagaaacccagaaccaggtccttgacacgacagcgacggtctctcctcccatctccgcccacaaccacgcaggcgaagaccacaaagaattggtttcagaggattccactccagtggttcagatgcaaaacatcaagggaaaggacacaatgccaaccacagtgacgggtgtaccaacaaccacaccctctccatttccaatcaatgctcgcaacactgatcataccaaatcatttatcggcctggaggggccccaagaagaccacagcaccacacagcctgccaagaccaccagccaaccaaccaacagcacagaatcgacgacactaaacccaacatcagagccctccagtagaggcacgggaccatccagccccacggtccccaacaccacagaaagccacgccgaacttggcaagacaaccccaaccacactcccagaacagcacactgccgccagtgccattccaagagccgtgcaccccgacgaactcagtggacctggcttcctgacgaacacaatacggggggtgacaaatctcctgacaggatccagaagaaagcgaagggatgtcactcccaatacacaacccaaatgcaacccaaacctgcactattggacagccttggatgagggtgctgccataggtttagcctggataccatacttcgggccagcagctgagggaatttacactgaaggcataatggagaatcaaaatggattgatctgtggattgaggcagctggccaacgaaacgacacaagctcttcaattgttcttaagggcaactactgagttgcgtacattctctatactaaatcggaaagcaatagacttcttgctccaaagatggggaggaacatgtcacattctagggcctgattgttgcattgaaccccaagattggaccaaaaatatcactgataaaattgatcaaataatccatgactttgtcgataataatcttccaaatcagaatgatggcagcaactggtggactggatggaaacaatgggttcctgctggaataggaatcacaggagtaatcattgctattattgctttgctgtgcatttgcaaattcatgctttgaactaatatagcatcatactttagaattctagaccaggcgcctggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 7073 bp, Artificial Sequence
  Description of Artificial SequencePlasmid containing DNA for GP of Ebola Virus, Sudan strain
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagctagatgcatgctcgagcggccgccagtgtgatggatatctgcagaattctatcttcaggatctcgccatggagggtcttagcctactccaattgcccagagataaatttcgaaaaagctctttctttgtttgggtcatcatcttatttcaaaaggccttttccatgcctttgggtgttgtgaccaacagcactttagaagtaacagagattgaccagctagtctgcaaggatcatcttgcatcaactgaccagctgaaatcagttggtctcaacctcgaggggagcggagtatctactgatatcccatctgcgacaaagcgttggggcttcagatctggtgtgcctccccaagtggtcagctatgaagcaggagaatgggctgaaaattgctacaatcttgaaataaagaaaccggacgggagcgaatgcttacccccaccgccggatggtgtcagaggctttccaaggtgccgctatgttcacaaagcccaaggaaccgggccctgcccgggtgactatgcctttcacaaggatggagctttcttcctctatgacaggctggcttcaactgtaatttacagaggagtcaattttgctgagggggtaatcgcattcttgatattggctaaaccaaaggaaacgttccttcaatcaccccccattcgagaggcagcaaactacactgaaaatacatcaagttactatgccacatcctacttggagtacgaaatcgaaaattttggtgctcaacactccacgacccttttcaaaattaacaataatacttttgttcttctggacaggccccacacgcctcagttccttttccagctgaatgataccattcaacttcaccaacagttgagcaacacaactgggaaactaatttggacactagatgctaatatcaatgctgatattggtgaatgggctttttgggaaaataaaaaaatctctccgaacaactacgtggagaagagctgtctttcgaaactttatcgctcaacgagacagaagacgatgatgcgacatcgtcgagaactacaaagggaagaatctccgaccgggccaccaggaagtattcggacctggttccaaaggattcccctgggatggtttcattgcacgtaccagaaggggaaacaacattgccgtctcagaattcgacagaaggtcgaagagtagatgtgaatactcaggaaactatcacagagacaactgcaacaatcataggcactaacggtaacaacatgcagatctccaccatcgggacaggactgagctccagccaaatcctgagttcctcaccgaccatggcaccaagccctgagactcagacctccacaacctacacaccaaaactaccagtgatgaccaccgaggaaccaacaacaccaccgagaaactctcctggctcaacaacagaagcacccactctcaccaccccagagaatataacaacagcggttaaaactgtttgggcacaagagtccacaagcaacggtctaataacttcaacagtaacaggtattcttgggagccttggacttcgaaaacgcagcagaagacaagttaacaccagggccacgggtaaatgcaatcccaacttacactactggactgcacaagaacaacataatgctgctgggattgcctggatcccgtactttggaccgggtgcagaaggcatatacactgaaggccttatgcacaaccaaaatgccttagtctgtggactcagacaacttgcaaatgaaacaactcaagctctgcagcttttcttaagggccacgacggagctgcggacatataccatactcaataggaaggccatagatttccttctgcgacgatggggcgggacatgtaggatcctgggaccagattgttgcattgagccacatgattggaccaaaaacatcactgataaaatcaaccaaatcatccatgatttcatcgacaaccctttacccaatcaggataatgatgataattggtggacgggctggagacagtggatccctgcaggaataggcattactggaattattattgcaatcattgctcttctttgcgtctgcaagctgctttgttgaatatcagaattccagcactggcggccgttactagtggatccgagctcggatccaagctctagaccaggcgcctggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 7285 bp, Artificial Sequence
  Description of Artificial SequencePlasmid containing DNA of GP of Ebola Virus, Zaire strain
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaggcgcctggatcgatccgcgatgaagattaagccgacagtgagcgtaatcttcatctctcttagattatttgttttccagagtaggggtcgtcaggtccttttcaatcgtgtaaccaaaataaactccactagaaggatattgtggggcaacaacacaatgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttagtttttcttcagattgcttcatggaaaagctcagcctcaaatcaatgaaaccaggatttaattatatggattacttgaatctaagattacttgacaaatgataatataatacactggagctttaaacatagccaatgtgattctaactcctttaaactcacagttaatcataaacaaggtttggtaccgagctcgaattatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 7272 bp, Artificial Sequence
  Description of Artificial SequencePlasmid containing DNA for soluble GP of Ebola Virus, Zaire strain
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaggcgcctggatcgaattgatgaagattaagccgacagtgagcgtaatcttcatctctcttagattatttgttttccagagtaggggtcgtcaggtccttttcaatcgtgtaaccaaaataaactccactagaaggatattgtggggcaacaacacaatgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttagtttttcttcagattgcttcatggaaaagctcagcctcaaatcaatgaaaccaggatttaattatatggattacttgaatctaagattacttgacaaatgataatataatacactggagctttaaacatagccaatgtgattctaactcctttaaactcacagttaatcataaacaaggtttggaattgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc